(19) United States
(12) Patent Application Publication
(10) Pub. No.: US 2023/0070636 A1
(43) Pub. Date: Mar. 9, 2023

(54) APPARATUS AND METHOD FOR ESTIMATING BLOOD PRESSURE

(71) Applicant: SAMSUNG ELECTRONICS CO., LTD., Suwon-si (KR)

(72) Inventors: Jae Min KANG, Seoul (KR); Seung Woo NOH, Seongnam-si (KR); Sang Yun PARK, Hwaseong-si (KR); Jin Woo CHOI, Ansan-si (KR)

(73) Assignee: SAMSUNG ELECTRONICS CO., LTD., Suwon-si (KR)

(21) Appl. No.: 17/541,336

(22) Filed: Dec. 3, 2021

(30) Foreign Application Priority Data
Sep. 3, 2021 (KR) 10-2021-0117495

(51) Int. Cl. A61B 5/021; A61B 5/022; A61B 5/00

(52) U.S. Cl. CPC A61B 5/02116; A61B 5/02225; A61B 5/02125; A61B 5/681

(57) ABSTRACT

An apparatus for estimating blood pressure is provided. The apparatus may include a pulse wave sensor configured to measure from an object, a plurality of pulse wave signals having different wavelengths, a force sensor configured to measure a contact force applied by the object to the pulse wave sensor, and a processor configured to extract, from the plurality of pulse wave signals, at least one similarity feature indicating a similarity between the plurality of pulse wave signals, and estimate the blood pressure based on the similarity and the contact force that is measured at a point in time at which the at least one similarity feature is extracted.

### CROSS-REFERENCE TO RELATED APPLICATION(S)

This application claims priority to Korean Patent Application No. 10-2021-0117495, filed on Sep. 3, 2021, in the Korean Intellectual Property Office, the disclosure of which is incorporated by reference herein in its entirety.

### BACKGROUND

### 1. Field

Apparatuses and methods consistent with example embodiments relate to estimating blood pressure without an inflatable arm cuff.

### 2. Description of Related Art

Generally, methods of non-invasively measuring blood pressure without damaging a human body include a method to measure blood pressure by measuring a cuff-based pressure and a method to estimate blood pressure by measuring a pulse wave without the use of a cuff.

A Korotkoff-sound method is one of cuff-based blood pressure measurement methods, in which a pressure in a cuff wound around an upper arm is increased and blood pressure is measured by listening to the sound generated in the blood vessel through a stethoscope while decreasing the pressure. Another cuff-based blood pressure measurement method is an oscillometric method using an automated machine, in which a cuff is wound around an upper arm, a pressure in the cuff is increased, a pressure in the cuff is continuously measured while the cuff pressure is gradually decreased, and blood pressure is measured based on a point where a change in a pressure signal is large.

Cuffless blood pressure measurement methods generally include a method of measuring blood pressure by calculating a pulse transit time (PTT) and a method a pulse wave analysis (PWA) method of estimating blood pressure by analyzing a shape of a pulse wave.

### SUMMARY

According to an aspect of an example embodiment, an apparatus for estimating blood pressure may include: a pulse wave sensor configured to measure from an object, a plurality of pulse wave signals having different wavelengths; a force sensor configured to measure a contact force applied by the object to the pulse wave sensor; and a processor configured to extract, from the plurality of pulse wave signals, at least one similarity feature indicating a similarity between the plurality of pulse wave signals, and estimate the blood pressure based on the similarity and the contact force that is measured at a point in time at which the at least one similarity feature is extracted.

The pulse wave sensor may include one or more light sources configured to emit light of the different wavelengths to the object, and one or more detectors configured to detect the light of the different wavelengths reflected or scattered from the object.

The processor may be further configured to: obtain a plurality of beat pulses by beat parsing each of the plurality of pulse wave signals; normalize each of the plurality of beat pulses; and extract, as the at least one similarity feature, at least one of a minimum value of a time-delay between the plurality of beat pulses, and a maximum value of a degree of sameness of a waveform shape between the plurality of beat pulses.

The processor may be further configured to: extract, as the at least one similarity feature, at least one of an onset point, an offset point, a max slope point, and a tangent max point from each of the plurality of beat pulses; and obtain a delay time by calculating at least one of a first time difference between the onset points extracted from each of the plurality of beat pulses, a second time difference between the offset points extracted from each of the plurality of beat pulses, a third time difference between the max slope points extracted from each of the plurality of beat pulses, and a fourth time difference between the tangent max points extracted from each of the plurality of beat pulses.

The processor may be further configured to obtain the degree of sameness of the waveform shape based on an area of a waveform of any one of the plurality of beat pulses and a mean absolute error (MAE) between the plurality of beat pulses.

The plurality of beat pulses may be obtained from a first pulse wave signal of a green light wavelength and a second pulse wave signal of a red light wavelength, among the plurality of pulse wave signals.

The point in time at which the at least one similarity feature is extracted may include at least one of a first point in time at which a minimum value of a time delay is obtained in each of the plurality of pulse wave signals and a second point in time at which a maximum value of a degree of sameness of a waveform shape is obtained in each of the plurality of pulse wave signals.

The processor may be further configured to estimate the blood pressure by combining the similarity between the plurality of pulse wave signals and the contact force at the point in time at which the at least one similarity feature is extracted through a predefined blood pressure estimation model.

The processor may be further configured to: generate an oscillometric envelope based on the plurality of pulse wave signals and the contact force measured by the force sensor; obtain one or more additional features using the generated oscillometric envelope, and estimate the blood pressure by combining the similarity between the plurality of pulse wave signals, the contact force at the point in time at which the at least one similarity feature is extracted, and the one or more additional features through a predefined blood pressure estimation model.

The one or more additional features may include at least one of a maximum point of an amplitude of the oscillometric envelope, the contact force corresponding to the maximum point, and the contact force corresponding to a predetermined ratio of the maximum point.

The apparatus may further include a display configured to output guide information regarding the contact force between the object and a sensor surface in response to receiving a request for estimating the blood pressure.

The guide information may include information for inducing the object to gradually increase the contact force applied to the sensor surface or to gradually decrease the contact force from a pressure intensity greater than or equal to a predetermined threshold.

According to an aspect of another example embodiment, there is provided a method of estimating blood pressure, the method including: measuring from an object, a plurality of pulse wave signals having different wavelengths; measuring a contact force applied by the object to a pulse wave signal; extracting, from the plurality of pulse wave signals, at least one similarity feature indicating a similarity between the plurality of pulse wave signals; obtaining the contact force at a point in time at which the at least one similarity feature is extracted; and estimating the blood pressure based on the similarity between the plurality of pulse wave signals and the contact force at the point in time at which the at least one similarity feature is extracted.

The extracting the at least one similarity feature may include: obtaining a plurality of beat pulses by beat parsing each of the plurality of pulse wave signals; normalizing each of the plurality of beat pulses; and extracting, as the at least one similarity feature, at least one of a minimum value of a time-delay between the plurality of beat pulses, and a maximum value of a degree of sameness of waveform shape between the plurality of beat pulses.

The extracting the at least one similarity feature may include: extracting, as the at least one similarity feature, at least one of an onset point, an offset point, a max slope point, and a tangent max point from each of the plurality of beat pulses; and obtaining a delay time by calculating at least one of a first time difference between the onset points extracted from each of the plurality of beat pulses, a second time difference between the offset points extracted from each of the plurality of beat pulses, a third time difference between the max slope points extracted from each of the plurality of beat pulses, and a fourth time difference between the tangent max points extracted from each of the plurality of beat pulses.

The extracting the at least one similarity feature may include obtaining the degree of sameness of the waveform shape based on an area of a waveform of any one of the plurality of beat pulses of and an mean absolute error (MAE) between the plurality of beat pulses.

The plurality of beat pulses may be obtained from a first pulse wave signal of a green light wavelength and a second pulse wave signal of a red light wavelength, among the plurality of pulse wave signals.

The point in time at which the at least one similarity feature is extracted may include at least one of a first point in time at which a minimum value of a time delay is obtained in each of the plurality of pulse wave signals and a second point in time at which a maximum value of a degree of sameness of a waveform shape is obtained in each of the plurality of pulse wave signals.

The estimating the blood pressure may include estimating the blood pressure by combining the similarity between the plurality of pulse wave signals and the contact force at the point in time at which the at least one similarity feature is extracted through a predefined blood pressure estimation model.

The estimating the blood pressure may include: generating an oscillometric envelope based on the plurality of pulse wave signals and the contact force; obtaining one or more additional features using the generated oscillometric envelope; and estimating the blood pressure by combining the similarity between the plurality of pulse wave signals, the contact force at the point in time at which the at least one similarity feature is extracted, and the one or more additional features through a predefined blood pressure estimation model.

### BRIEF DESCRIPTION OF THE DRAWINGS

The above and/or other aspects will be more apparent by describing certain example embodiments, with reference to the accompanying drawings, in which:

FIG. 1 is a block diagram of an apparatus for estimating blood pressure according to an exemplary embodiment;

FIGS. 2 and 3 are diagrams for explaining a method of extracting a minimum value of a time delay between pulse wave signals;

FIGS. 4 and 5 are diagrams for explaining a method of extracting a maximum value of a degree of sameness of waveform shape between pulse wave signals;

FIGS. 6A and 6B are diagrams for explaining a method of obtaining additional feature values using an oscillometric envelope;

FIG. 7 is a block diagram illustrating an apparatus for estimating blood pressure according to another exemplary embodiment;

FIG. 8 is a flowchart illustrating a method of estimating blood pressure according to an exemplary embodiment;

FIGS. 9 to 11 are diagrams illustrating examples of an electronic device including an apparatus for estimating blood pressure.

### DETAILED DESCRIPTION

Example embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the example embodiments. However, it is apparent that the example embodiments can be practiced without those specifically defined matters. Also, well-known functions or constructions are not described in detail since they would obscure the description with unnecessary detail.

It will be understood that, although the terms first, second, etc. may be used herein to describe various elements, these elements should not be limited by these terms. These terms are only used to distinguish one element from another. Also, the singular forms are intended to include the plural forms as well, unless the context clearly indicates otherwise. In the specification, unless explicitly described to the contrary, the word “comprise” and variations such as “comprises” or “comprising,” will be understood to imply the inclusion of stated elements but not the exclusion of any other elements. Terms such as “unit” and “module” denote units that process at least one function or operation, and they may be implemented by using hardware, software, or a combination of hardware and software.

Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, all of a, b, and c, or any variations of the aforementioned examples.

Hereinafter, an apparatus and method for estimating blood pressure will be described in detail with reference to the accompanying drawings. Various embodiments of an apparatus for estimating blood pressure described hereinafter may be mounted on a variety of devices, such as portable wearable devices, smart devices, or the like. For example, the variety of devices may include various types of wearable devices, such as a smartwatch worn on a wrist, a smart band type wearable device, a headphone-type wearable device, and a hair band type wearable device, and mobile devices, such as a smartphone, a tablet personal computer (PC), earbuds, etc.

In the following embodiment, blood pressure estimation will be described as an example, but the present disclosure is not limited thereto, and other bio-information using a pulse wave signal, for example, heart rate, vascular age, arterial stiffness, aortic pressure waveform, blood vessel elasticity, stress index, fatigue level, skin elasticity, skin age, and the like, may be estimated.

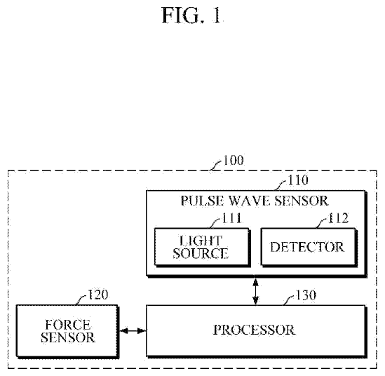

FIG. 1 is a block diagram of an apparatus for estimating blood pressure according to an exemplary embodiment.

Referring to FIG. 1, an apparatus 100 for estimating blood pressure according to an exemplary embodiment may include a pulse wave sensor 110, a force sensor 120, and a processor 130.

The pulse wave sensor 110 may measure a pulse wave signal including a photoplethysmography (PPG) signal from an object and measure a plurality of pulse wave signals having different wavelengths. In this case, the different wavelengths may include green, blue, red, and infrared wavelengths.

The pulse wave sensor 110 may include one or more light sources 111 configured to emit light of different wavelengths to the object and one or more detectors 112 configured to detect light of different wavelengths reflected or scattered from the object. The light source 111 may include a light emitting diode (LED), a laser diode, and a phosphor, but is not limited thereto. In addition, the detector 112 may include a photodiode, a photo transistor (PTr), an image sensor (e.g., a complementary metal oxide semiconductor (CMOS) image sensor), etc. However, the examples of the detector are not limited thereto. The pulse wave sensor 110 may be configured with an array of one or more light sources 111 and/or an array of one or more detectors 112 to measure two or more pulse wave signals. In this case, the one or more light sources 111 may emit light of different wavelengths from each other, and each light source 111 may be disposed at a different distance from the detector 112.

The force sensor 120 may measure a contact force applied by the object to the pulse wave sensor 110. The force sensor 120 may measure a contact force exerted to the pulse wave sensor 110 when the object (which may be a body part of a user) contacts the pulse wave sensor 110 and gradually increases a pressing force or gradually decreases an applied force that is greater than or equal to a threshold. The force sensor 120 may be disposed on an upper or lower side of the pulse wave sensor 110. The force sensor 120 may include a strain gauge or the like, and may be configured as a single force sensor or an array of force sensors. In this case, the force sensor 120 may be included in a pressure sensor in which the force sensor 120 and an area sensor are combined, or may be implemented as a pressure sensor in the form of an air bag, a force matrix sensor capable of measuring a force for each pixel, or the like.

The processor 130 may be electrically connected to the pulse wave sensor 110 and/or the force sensor 120, and may control the pulse wave sensor 110 and/or the force sensor 120 in response to a request for estimating bio-information.

When the pulse wave signals of a plurality of wavelengths are received from the pulse wave sensor 110, the processor 130 may pre-process each pulse wave signal. For example, the processor 130 may perform preprocessing on the received pulse wave signal, such as filtering for removing noise from the received pulse wave signal, amplification of the bio-signal, or converting the pulse wave signal into a digital signal.

Also, the processor 130 may extract similarity of pulse waves between the plurality of measured pulse wave signals, and estimate the blood pressure based on the extracted similarity and a contact force at a point in time related to the similarity.

The similarity of pulse waves refers to a feature value that can be obtained by combining different wavelengths, for example, an infrared wavelength and a green wavelength, and is an indicator of a point at which different pulse wave signals with different penetration depths exhibit similar characteristics where the patterns thereof become similar over time when skin and tissues are pressed by compression during the process of pressing the surface. For example, the similarity of pulse waves may include a minimum value of a time delay between corresponding beat pulses of each of the plurality of pulse wave signals and/or a maximum value of a degree of sameness of waveform shape between the corresponding beat pulses of each pulse wave signal.

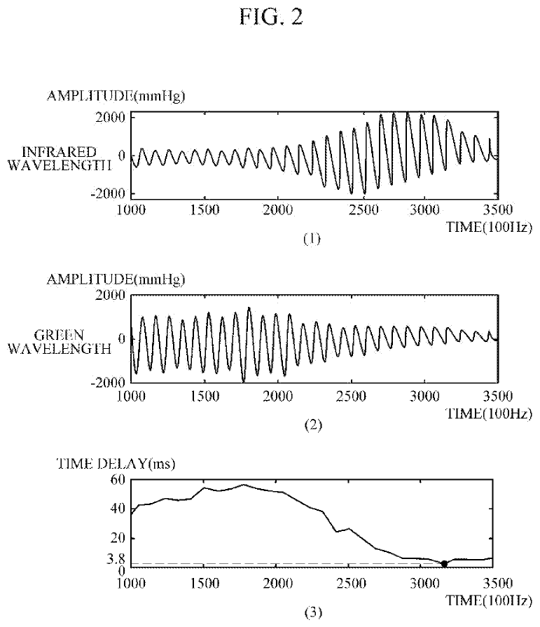

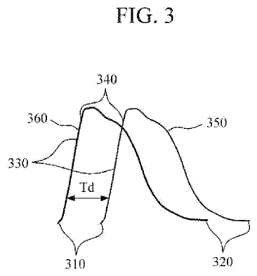

FIGS. 2 and 3 are diagrams for explaining a method of extracting a minimum value of a time delay between pulse wave signals.

Referring to FIG. 2, when the user gradually increases and decreases contact pressure while touching the pulse wave sensor 110 with a finger, the waveform of a pulse wave signal having an infrared wavelength that penetrates relatively deeply into the skin varies with time as shown in graph (1), and the waveform of a pulse wave signal having a green wavelength that penetrates shallowly into the skin surface varies with time as shown in graph (2).

The processor 130 may obtain a plurality of beat pulses by beat parsing the pulse wave signal of each wavelength and normalize each of the obtained beat pulses.

Referring to FIG. 3, the processor 130 may extract corresponding onset points 310, corresponding offset points 320, corresponding max slope points 330, corresponding tangent max points 340, and the like for a normalized infrared wavelength beat pulse 360 and a normalized green wavelength beat pulse 350, and may obtain a time delay by calculating a time difference Td between the extracted corresponding points. Graph (3) in FIG. 2 connects the time delays between all beat pulses of the pulse wave signal having the infrared wavelength and the pulse wave signal having the green wavelength, and, for example, 3.8 ms, which is a minimum value of the time delay, may be extracted as a similarity of pulse waves.

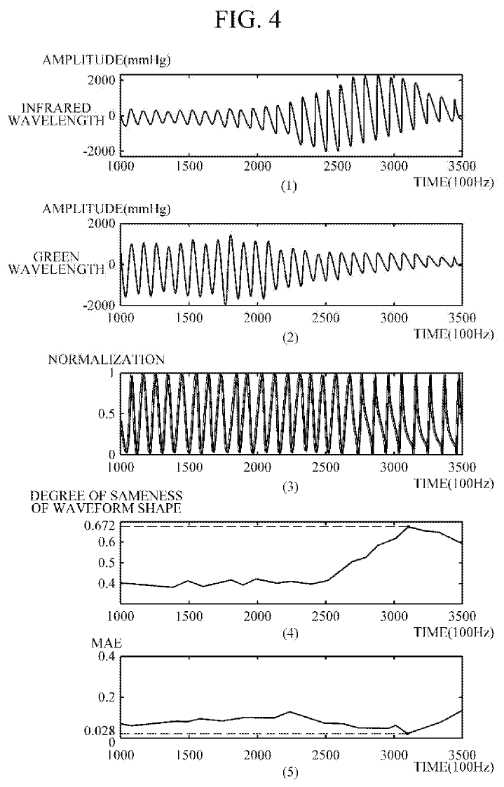

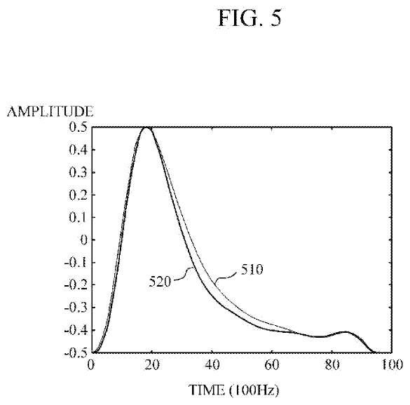

FIGS. 4 and 5 are diagrams for explaining a method of extracting a maximum value of a degree of sameness of waveform shape between pulse wave signals.

Referring to FIG. 4, when the user gradually increases and decreases the contact pressure while touching the pulse wave sensor 110 with a finger, the waveform of a pulse wave signal having the infrared wavelength that penetrates relatively deeply into the skin varies with time as shown in graph (1), and the waveform of a pulse wave signal having the green wavelength that penetrates shallowly into the skin surface varies with time as shown in graph (2).

The processor 130 may obtain a plurality of beat pulses by beat parsing each of the plurality of pulse wave signals and normalize each of the beat pulses, and superimposition of the normalized beat pulses is displayed as shown in graph (3) in FIG. 4.

The processor 130 may obtain a degree of sameness of a waveform of the pulse wave signals based on the area of the normalized beat pulse of each pulse wave signal. For example, the processor 130 may obtain the degree of sameness of the waveform based on the area of the waveform of any one of the corresponding normalized beat pulses of each pulse wave signal and/or a mean absolute error (MAE) between the corresponding beat pulses.

In this case, any one bit pulse may be a bit pulse having a relatively short wavelength (e.g., green wavelength). Also, the processor 130 may determine an MAE between the corresponding beat pulses of each pulse wave signal and calculate the degree of sameness of waveform between the corresponding beat pulse waveforms using the area Pulseₐᵣₑₐ of any one pulse waveform and the MAE as shown in Equation 1 below. In this case, the MAE represents the mean of the sum of absolute values for the difference in amplitude values obtained at the same point in time for the corresponding beat pulse waveforms.

Degree of sameness of waveform=(1−Pulseₐᵣₑₐ)*(1−MAE) (1)

Referring to FIG. 5, for example, the area Pulseₐᵣₑₐ may be the area below the waveform of a normalized green wavelength beat pulse 510, and the MAE represents the mean of the sum of absolute values for the difference in amplitude values obtained at the same point in time for a normalized infrared wavelength beat pulse 520 and the normalized green wavelength beat pulse 510.

Graph (5) in FIG. 4 connects MAEs between all beat pulses of the pulse wave signal having the infrared wavelength and the pulse wave signal having the green wavelength, and graph (4) connects the degree of sameness of waveform shape between all beat pulses of the pulse wave signal having the infrared wavelength and the pulse wave signal having the green wavelength. For example, 0.0672, which is a maximum value of the degree of sameness of waveform shape at 0.028 which is the minimum MAE, may be extracted as the similarity of pulse waves.

In general, the shape of the beat pulses of the normalized wavelengths of the pulse wave signal having the infrared wavelength and the pulse wave signal having the green wavelength changes from a blunt shape to a pointed shape with time. It can be seen that the degree of sameness of shape appears maximum in the pointed portion of the waveform.

When the similarity of pulse waves is determined, the processor 130 may further extract a contact force at the point in time related to the similarity among the contact forces received from the force sensor 120 as a feature for blood pressure estimation. For example, a contact force at a point in time at which the time delay in the similarity of pulse waves is minimum and/or a contact force at a point in time at which the degree of sameness of waveform shape is maximum may be extracted.

The processor 130 may estimate blood pressure by combining the similarity of pulse waves and the contact force at the point in time related to the similarity through a predefined blood pressure estimation model. That is, blood pressure may be estimated based not only one the similarity of pulse waves but also on the contact force at the point in time at which the similarity of pulse waves occurs as the features. In this case, the predefined blood pressure estimation model may be defined as various linear or non-linear combination functions, such as addition, subtraction, division, multiplication, logarithmic value, regression equation, and the like, with no specific limitation. For example, Equation 2 below represents a simple linear function.

y=aƒ₁+bƒ₂+c [Equation 2]

Here, y represents blood pressure to be obtained, for example, diastolic blood pressure, systolic blood pressure, and mean blood pressure, or the like. ƒ₁ represents a first feature value. ƒ₂ represents a second feature value. For example, the first feature value may be similarity of pulse waves between pulse wave signals having different wavelengths and the second feature value may be a contact force at a point in time at which the similarity occurs. a, b, and c are values obtained in advance through a preprocessing process, and may be defined differently according to the type of bio-information to be obtained and the user characteristics. Here, ƒ₁ may be any one of the first feature values or a value obtained by combining two or more of the first feature values. Here, ƒ₂ may be any one of the second feature values or a value obtained by combining two or more of the second feature values.

In addition, the processor 130 may generate an oscillometric envelope based on the pulse wave signal and the contact force measured by the force sensor, obtain one or more additional features using the generated oscillometric envelope, and estimate the blood pressure by combining the similarity of pulse waves, the contact force at the point in time related to the similarity, and the one or more additional features through the predefined blood pressure estimation model.

For example, the processor 130 may extract a peak-to-peak point at each measurement time of the pulse wave signal, and plot the extracted peak-to-peak point on the basis of a contact force corresponding to each measurement time to obtain an oscillometric envelope representing the contact force versus the pulse wave at each measurement time.

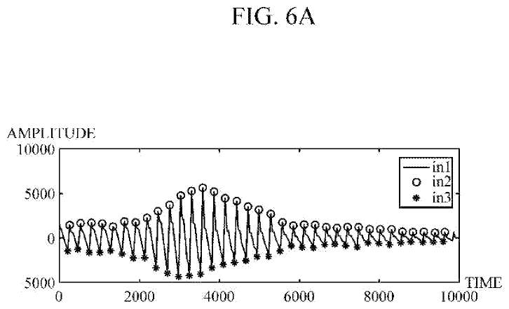

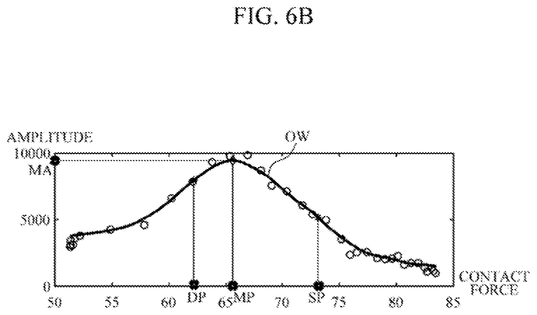

Referring to FIG. 6A, a pulse wave signal is obtained by gradually increasing contact pressure while a user touches the pulse wave sensor 110 with a finger or by gradually decreasing contact pressure when a user touches the pulse wave sensor 110 with a pressure intensity greater than or equal to a predetermined threshold. The processor 130 may extract the peak-to-peak point by subtracting an amplitude value in3 of a negative (−) point from an amplitude value in2 of a positive (+) point of the waveform envelope in1 at each measurement time of the obtained pulse wave signal.

Referring to FIG. 6B, the processor 130 may obtain an oscillometric envelope OW by plotting a peak-to-peak amplitude at each measurement time point based on a contact force at the same measurement time point as the peak-to-peak amplitude. The processor 130 may obtain one or more additional features from the obtained oscillometric envelope OW. Referring to FIG. 6B, the processor 130 may include a maximum amplitude MA at a maximum peak point in the oscillometric envelope OW as a feature, and may obtain, as additional features, a contact force MP at the maximum peak point, and contact forces SP and DP located to the left and right of the contact force MP of the maximum peak point and having a predetermined ratio (e.g., 0.5 to 0.7) to the contact force MP, and the like.

For example, the processor 130 may estimate blood pressure through the predefined blood pressure estimation model by using the additional features obtained using the oscillometric envelope of the infrared wavelength or green wavelength, the similarity of pulse waves between the infrared wavelength and the green wavelength, and the contact force at a point in time at which the similarity occurs. In this case, the predefined blood pressure estimation model may be defined as various linear or non-linear combination functions, such as addition, subtraction, division, multiplication, logarithmic value, regression equation, and the like, with no specific limitation.

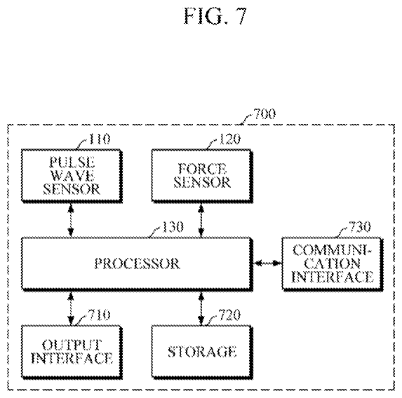

FIG. 7 is a block diagram illustrating an apparatus for estimating blood pressure according to another exemplary embodiment.

Referring to FIG. 7, an apparatus 700 for estimating blood pressure according to another exemplary embodiment may further include at least one of an output interface 710, a storage 720, and a communication interface 730, in addition to a pulse wave sensor 110, a force sensor 120 and a processor 130. The pulse wave sensor 110, the force sensor 120, and the processor 130 are described with reference to FIG. 1, and hence detailed descriptions thereof will not be reiterated.

The output interface 710 may output a pulse wave signal and a contact force obtained by the pulse wave sensor and the force sensor 120 under the control of the processor 130, and various processing results of the processor 130. In addition, upon receiving a request for estimating blood pressure, the output interface 730 may output guide information regarding a contact force between an object and a sensor part. In this case, the guide information may include information for inducing the object to gradually increase the contact force applied to the sensor part or to gradually decrease a contact force from a pressure intensity greater than or equal to a predetermined threshold.

The output interface 710 may visually output an estimated blood pressure value and/or the guide information through a display, or may output the same through a speaker, a haptic sensor, or the like in a non-visual manner, such as voice, vibration, tactile sensation, etc. A display area may be divided into two or more areas, in which the pulse wave signal, the contact force, and the like, which are used for estimating bio-information, may be output in various forms of graphs in a first area; and an estimated blood pressure value may be output in a second area. In this case, if an estimated blood pressure value falls outside a normal range, the output interface 710 may output warning information in various manners, such as highlighting an abnormal value in red and the like, displaying the abnormal value along with a normal range, outputting a voice warning message, adjusting a vibration intensity, and the like.

The storage 720 may store processing results of the pulse wave sensor 110 and the processor 130. In addition, the storage 720 may store various types of reference information for estimating bio-information. For example, the reference information may include user characteristic information, such as a user's age, gender, health condition, and the like. In addition, the reference information may include various types of information, such as a blood pressure estimation model, blood pressure estimation criteria, a reference contact force, a reference feature value, and the like, but is not limited thereto.

In particular, the storage 720 may include at least one type of storage medium of a flash memory type, a hard disk type, a multimedia card micro type, a card type memory (for example, secure digital (SD) or extreme digital (XD) memory), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk, but is not limited thereto.

The communication interface 730 may communicate with an external device under the control of the processor 130 to transmit and receive various data using wired or wireless communication techniques. For example, the communication interface 730 may transmit a blood pressure estimation result to the external device and receive various types of reference information required for blood pressure estimation from the external device. In this case, the external device may include an information processing device, such as a cuff-type blood pressure measurement device, a smartphone, earbuds, a tablet PC, a desktop PC, a notebook PC, and the like. In addition, the communication interface 730 may transmit guide information regarding contact of the object which is generated by the processor 130 during the measurement of the pulse wave signal to the external device, so that the guide information can be displayed on a display of the external device.

In this case, the communication techniques may Bluetooth communication, Bluetooth low energy (BLE) communication, near field communication (NFC), wireless local access network (WLAN) communication, ZigBee communication, infrared data association (IrDA) communication, Wi-Fi Direct (WFD) communication, ultra-wideband (UWB) communication, Ant+ communication, Wi-Fi communication, radio frequency identification (RFID) communication, 3G communication, 4G communication, and/or 5G communication. However, the communication techniques are not limited thereto.

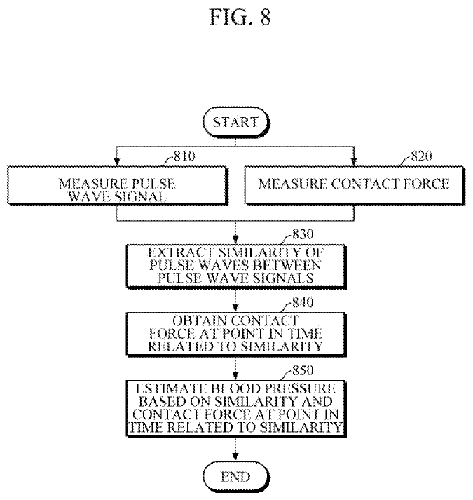

FIG. 8 is a flowchart illustrating a method of estimating blood pressure according to an exemplary embodiment.

The method of FIG. 8 may be performed by the apparatuses 100 and 700 for estimating blood pressure according to the exemplary embodiments of FIGS. 1 and 7. The method is described in detail above, and hence will be briefly described hereinafter.

First, the apparatus for estimating blood pressure may measure a plurality of pulse wave signals having different wavelengths from an object using a pulse wave sensor in operation 810.

Also, the apparatus may measure a contact force applied by the object to the pulse wave sensor using a force sensor in operation 820.

Then, similarity of pulse waves between the plurality of measured pulse wave signals may be extracted in operation 830.

The similarity of pulse waves refers to a feature value that can be obtained by combining different wavelengths, for example, an infrared wavelength and a green wavelength, and is an indicator of a point at which different pulse wave signals with different penetration depths exhibit similar characteristics where the patterns thereof become similar over time when skin and tissues are pressed by compression during the process of pressing the surface. For example, the similarity of pulse waves may include a minimum value of a time delay between corresponding beat pulses of each of the plurality of pulse wave signals and/or a maximum value of a degree of sameness of waveform shape between the corresponding beat pulses of each pulse wave signal.

Here, the apparatus may extract at least one of an onset point, an offset point, a max slope point, and a tangent max point from each of the corresponding beat pulses of each pulse wave signal and obtain the delay time by calculating a time difference between the extracted corresponding points. Also, the apparatus may obtain the degree of sameness of waveform shape based on the area of the waveform of any one of the corresponding beat pulses of each pulse wave signal and an MAE between the corresponding beat pulses.

Thereafter, a contact force at a point in time related to the similarity of pulse waves may be obtained in operation 840. The point in time related to the similarity of pulse waves may include a point in time at which the minimum value of the time delay is obtained in the pulse wave signal and a point in time at which the maximum value of the degree of sameness of waveform shape is obtained.

Then, blood pressure may be estimated based on the similarity of pulse waves and the contact force at the point in time related to the similarity in operation 850. The blood pressure may be estimated by combining the similarity of pulse waves and the contact force at the point in time related to the similarity through a predefined blood pressure estimation model. In addition, the apparatus may generate an oscillometric envelope based on the pulse wave signal and the contact force measured by the force sensor, acquire one or more additional features using the generated oscillometric envelope, and estimate the blood pressure by combining the similarity of pulse waves, the contact force at the point in time related to the similarity, and the one or more additional features through a predefined blood pressure estimation model.

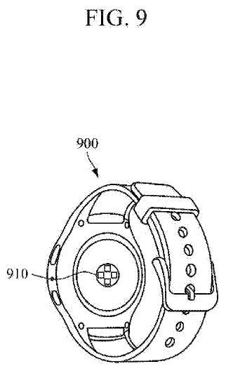

FIGS. 9 to 11 are diagrams illustrating examples of an electronic device including embodiments of an apparatus for estimating blood pressure.

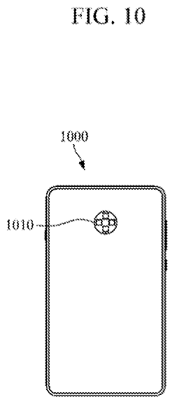

An electronic device may include a wearable device 900 of a smart watch type and a mobile device 1000, such as a smartphone, as shown in FIGS. 9 and 10. However, the electronic device is not limited to the aforementioned examples, and may include a smart band, smart glasses, a smart ring, a smart patch, a smart necklace, a tablet PC, etc. The electronic device may include the apparatus 100 or 700 for estimating blood pressure and all components of the apparatus 100 or 700 may be mounted in one electronic device, or separately mounted in two or more electronic devices.

Referring to FIG. 9, the electronic device may be configured as a watch-type wearable device 900 and may include a main body and a strap. A display may be provided on the front surface of the main body to display general application screens containing time information, received message information, etc. and/or a blood pressure estimation application screen containing object contact guide information, blood pressure estimation results, and the like. A sensor module 910 including a pulse wave sensor and a force sensor may be disposed on a rear surface of the main body to obtain a pulse wave signal and force/pressure for blood pressure estimation from a contacting area of a wrist of the user. In addition, a processor configured to guide the contact of the object or estimate blood pressure using data received from the sensor module 910, an output interface configured to output data generated by the processor to the display, a communication interface configured to communicate with other electronic devices to transmit and receive information, and the like may be included inside the main body.

Referring to FIG. 10, the electronic device may be implemented as a mobile device 1000 such as a smartphone.

The mobile device 1000 may include a housing and a display panel. The housing may form the outer appearance of the mobile device 1000. The display panel and cover glass may be sequentially arranged on a first surface of the main body, and the display panel may be exposed to the outside through the cover glass. A sensor module 1010, a camera module, and/or an infrared sensor may be disposed on a second surface of the main body. When a user executes an application or the like installed in the mobile device 1000 to request estimation of blood pressure, a pulse wave signal and a contact force may be measured from an object using the sensor module 1010. A processor configured to guide the contact of the object or estimate blood pressure using data received from the sensor module 1010, an output interface configured to output data generated by the processor to the display, a communication interface configured to communicate with other electronic devices to transmit and receive information, and the like may be included inside the main body.

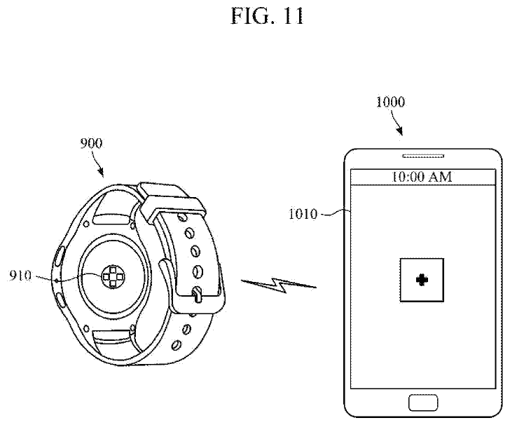

FIG. 11 is a diagram illustrating an example in which the watch-type wearable device 900 and the mobile device 1000 cooperate to estimate blood pressure. When the user estimates blood pressure using the wearable device 900, various types of related information may be displayed on the display screen of the mobile device 1000. Conversely, when blood pressure is estimated using the mobile device 1000, the related information may be displayed on the display screen of the wearable device 900. The wearable device 900 may transmit the guide information regarding the contact of the object which is generated by the processor to the mobile device 1000, so that the guide information can be output to the screen of the display of the mobile device 1000.

While not restricted thereto, an example embodiment can be embodied as computer-readable code on a computer-readable recording medium. The computer-readable recording medium is any data storage device that can store data that can be thereafter read by a computer system. Examples of the computer-readable recording medium include read-only memory (ROM), random-access memory (RAM), CD-ROMs, magnetic tapes, floppy disks, and optical data storage devices. The computer-readable recording medium can also be distributed over network-coupled computer systems so that the computer-readable code is stored and executed in a distributed fashion. Also, an example embodiment may be written as a computer program transmitted over a computer-readable transmission medium, such as a carrier wave, and received and implemented in general-use or special-purpose digital computers that execute the programs. Moreover, it is understood that in example embodiments, one or more units of the above-described apparatuses and devices can include circuitry, a processor, a microprocessor, etc., and may execute a computer program stored in a computer-readable medium.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An apparatus for estimating blood pressure, the apparatus comprising:
a pulse wave sensor configured to measure from an object, a plurality of pulse wave signals having different wavelengths;
a force sensor configured to measure a contact force applied by the object to the pulse wave sensor; and
a processor configured to extract, from the plurality of pulse wave signals, at least one similarity feature indicating a similarity between the plurality of pulse wave signals, and estimate the blood pressure based on the similarity and the contact force that is measured at a point in time at which the at least one similarity feature is extracted.

2. The apparatus of claim 1, wherein the pulse wave sensor comprises one or more light sources configured to emit light of the different wavelengths to the object, and one or more detectors configured to detect the light of the different wavelengths reflected or scattered from the object.

3. The apparatus of claim 1, wherein the processor is further configured to:
obtain a plurality of beat pulses by beat parsing each of the plurality of pulse wave signals;
normalize each of the plurality of beat pulses; and
extract, as the at least one similarity feature, at least one of a minimum value of a time-delay between the plurality of beat pulses, and a maximum value of a degree of sameness of a waveform shape between the plurality of beat pulses.

4. The apparatus of claim 3, wherein the processor is further configured to:
extract, as the at least one similarity feature, at least one of an onset point, an offset point, a max slope point, and a tangent max point from each of the plurality of beat pulses; and
obtain a delay time by calculating at least one of a first time difference between the onset points extracted from each of the plurality of beat pulses, a second time difference between the offset points extracted from each of the plurality of beat pulses, a third time difference between the max slope points extracted from each of the plurality of beat pulses, and a fourth time difference between the tangent max points extracted from each of the plurality of beat pulses.

5. The apparatus of claim 3, wherein the processor is further configured to obtain the degree of sameness of the waveform shape based on an area of a waveform of any one of the plurality of beat pulses and a mean absolute error (MAE) between the plurality of beat pulses.

6. The apparatus of claim 5, wherein the plurality of beat pulses are obtained from a first pulse wave signal of a green light wavelength and a second pulse wave signal of a red light wavelength, among the plurality of pulse wave signals.

7. The apparatus of claim 1, wherein the point in time at which the at least one similarity feature is extracted comprises at least one of a first point in time at which a minimum value of a time delay is obtained in each of the plurality of pulse wave signals and a second point in time at which a maximum value of a degree of sameness of a waveform shape is obtained in each of the plurality of pulse wave signals.

8. The apparatus of claim 1, wherein the processor is further configured to estimate the blood pressure by combining the similarity between the plurality of pulse wave signals and the contact force at the point in time at which the at least one similarity feature is extracted through a predefined blood pressure estimation model.

9. The apparatus of claim 1, wherein the processor is further configured to:
generate an oscillometric envelope based on the plurality of pulse wave signals and the contact force measured by the force sensor;
obtain one or more additional features using the generated oscillometric envelope, and
estimate the blood pressure by combining the similarity between the plurality of pulse wave signals, the contact force at the point in time at which the at least one similarity feature is extracted, and the one or more additional features through a predefined blood pressure estimation model.

10. The apparatus of claim 9, wherein the one or more additional features comprise at least one of a maximum point of an amplitude of the oscillometric envelope, the contact force corresponding to the maximum point, and the contact force corresponding to a predetermined ratio of the maximum point.

11. The apparatus of claim 1, further comprising a display configured to output guide information regarding the contact force between the object and a sensor surface in response to receiving a request for estimating the blood pressure.

12. The apparatus of claim 11, wherein the guide information comprises information for inducing the object to gradually increase the contact force applied to the sensor surface or to gradually decrease the contact force from a pressure intensity greater than or equal to a predetermined threshold.

13. A method of estimating blood pressure, the method comprising:
measuring from an object, a plurality of pulse wave signals having different wavelengths;
measuring a contact force applied by the object to a pulse wave signal;
extracting, from the plurality of pulse wave signals, at least one similarity feature indicating a similarity between the plurality of pulse wave signals;
obtaining the contact force at a point in time at which the at least one similarity feature is extracted; and
estimating the blood pressure based on the similarity between the plurality of pulse wave signals and the contact force at the point in time at which the at least one similarity feature is extracted.

14. The method of claim 13, wherein the extracting the at least one similarity feature comprises:
obtaining a plurality of beat pulses by beat parsing each of the plurality of pulse wave signals;
normalizing each of the plurality of beat pulses; and
extracting, as the at least one similarity feature, at least one of a minimum value of a time-delay between the plurality of beat pulses, and a maximum value of a degree of sameness of waveform shape between the plurality of beat pulses.

15. The method of claim 14, wherein the extracting the at least one similarity feature comprises:
extracting, as the at least one similarity feature, at least one of an onset point, an offset point, a max slope point, and a tangent max point from each of the plurality of beat pulses; and
obtaining a delay time by calculating at least one of a first time difference between the onset points extracted from each of the plurality of beat pulses, a second time difference between the offset points extracted from each of the plurality of beat pulses, a third time difference between the max slope points extracted from each of the plurality of beat pulses, and a fourth time difference between the tangent max points extracted from each of the plurality of beat pulses.

16. The method of claim 14, wherein the extracting the at least one similarity feature comprises obtaining the degree of sameness of the waveform shape based on an area of a waveform of any one of the plurality of beat pulses of and an mean absolute error (MAE) between the plurality of beat pulses.

17. The method of claim 16, wherein the plurality of beat pulses are obtained from a first pulse wave signal of a green light wavelength and a second pulse wave signal of a red light wavelength, among the plurality of pulse wave signals.

18. The method of claim 13, wherein the point in time at which the at least one similarity feature is extracted comprises at least one of a first point in time at which a minimum value of a time delay is obtained in each of the plurality of pulse wave signals and a second point in time at which a maximum value of a degree of sameness of a waveform shape is obtained in each of the plurality of pulse wave signals.

19. The method of claim 13, wherein the estimating the blood pressure comprises estimating the blood pressure by combining the similarity between the plurality of pulse wave signals and the contact force at the point in time at which the at least one similarity feature is extracted through a predefined blood pressure estimation model.

20. The method of claim 13, wherein the estimating the blood pressure comprises:
generating an oscillometric envelope based on the plurality of pulse wave signals and the contact force;
obtaining one or more additional features using the generated oscillometric envelope; and
estimating the blood pressure by combining the similarity between the plurality of pulse wave signals, the contact force at the point in time at which the at least one similarity feature is extracted, and the one or more additional features through a predefined blood pressure estimation model.
